(19) United States
(12) Patent Application Publication
(10) Pub. No.: US 2007/0077157 A1
(43) Pub. Date: Apr. 5, 2007

(54) Air compressor having improved valve device

(76) Inventor: Wen San Chou, Tainan Hsien (TW)

Correspondence Address: CHARLES E. BAXLEY, ESQ., 90 JOHN STREET, THIRD FLOOR, NEW YORK, NY, 10038, US

(21) Appl. No.: 11/204,523

(22) Filed: Aug. 16, 2005

(51) Int. Cl. A61M 1/00

(52) U.S. Cl. 417/437

(57) ABSTRACT

An air compressor includes a cylinder housing having an outlet tube, a piston slidably received in the cylinder housing and having a piston rod and a bore, and a motor coupled to the piston for moving the piston relative to the cylinder housing in a reciprocating action, in order to generate pressurized air. A valve device includes a blade member having one side secured to the piston, and the other side coupled to another blade member for increasing a resilience of the blade members, and includes an inner peripheral slot formed between the blade members, and a coupling member coupling the blade members together.

### BACKGROUND OF THE INVENTION

1. Field of the Invention

The present invention relates to an air compressor, and more particularly to an air compressor having an improved valve device for effectively controlling air to flow through the valve device or to flow into and out of the cylinder housing.

2. Description of the Prior Art

Typical air compressors comprise a cylinder housing attached or secured to a base and having a piston slidably disposed therein, and a motor secured to the base and coupled to the piston of the cylinder housing for actuating or driving the piston of the cylinder housing in a reciprocating action.

The applicant has developed various kinds of typical air compressors, such as U.S. Pat. No. 6,213,725 to Chou, which also comprises a piston slidably disposed within a cylinder housing and having an annular groove form therein, for loosely receiving a sealing ring and having one or more notches communicating with the annular groove of the piston.

Normally, the piston will be forced or driven to slide along or relative to the cylinder housing in a reciprocating action and in a great speed, such that the sealing ring may have a good chance to be damaged by the sliding friction from the piston and the cylinder housing after use, and such that the air intake function will become fail.

U.S. Pat. No. 6,280,163 to Chou discloses another typical air compressor which also comprises a piston slidably disposed within a cylinder housing, and a spring blade having one end secured to the piston and having the other end for selectively blocking an air aperture of the piston, in order to control the air to flow through the piston.

However, the spring blade is normally made of metal or steel materials, and may only be forced to slightly open the air aperture of the piston, such that only limited air may be allowed to flow through the piston at one time when the air aperture of the piston is partially opened by the spring blade. In addition, the spring blade may be easily bent or deformed relative to the one end thereof and thus may be become fail after use, and thus may not suitably block the air aperture of the piston when the spring blade has been bent or deformed.

U.S. Pat. No. 6,315,534 to Chou discloses a further typical air compressor which comprises a piston slidably disposed within a cylinder housing and having one or more air inlets formed therein, and a valve piece having a center portion secured to a central pin of the piston and having the outer peripheral portion for selectively blocking the air inlets of the piston.

However, similarly, the valve piece is normally made of metal or steel materials, and may only be forced to slightly open the air inlets of the piston, such that only limited air may be allowed to flow through the piston at one time when the air inlets of the piston is partially opened by the valve piece. Furthermore, the typical air compressor comprises a large number of parts or elements that may not be easily manufactured and assembled, and that may cost much money and expenses.

The present invention has arisen to mitigate and/or obviate the afore-described disadvantages of the conventional air compressors.

### SUMMARY OF THE INVENTION

The primary objective of the present invention is to provide an air compressor including an improved valve device for effectively controlling the air to flow through the valve device or to flow into and out of the cylinder housing.

In accordance with one aspect of the invention, there is provided an air compressor comprising a cylinder housing including a chamber formed therein, and including an outlet tube having a compartment formed therein and communicating with the chamber of the cylinder housing, for receiving pressurized air from the chamber of the cylinder housing, a piston slidably received in the chamber of the cylinder housing, and having a piston rod extended therefrom, and having a bore formed therein, and having a first side and a second side, a motor coupled to the piston rod, for moving the piston relative to the cylinder housing in a reciprocating action, in order to generate pressurized air, and a valve device including a first blade member having a first side secured to the first side of the piston, and having a second side opposite to the first side thereof, and a second blade member coupled to the second side of the first blade member, for blocking the bore of the piston, and for increasing a resilience of the second blade member of the valve device relative to the piston.

The valve device includes at least one aperture formed in the first side of the first blade member, and the piston includes at least one catch extended upwardly from the first side thereof and engaged into the aperture of the first blade member, for attaching the first blade member to the first side of the piston. The valve device includes a notch formed in the first side thereof, for increasing a resilience of the first blade member.

The valve device includes an inner peripheral slot formed therein, for forming the first blade member and the second blade member therein, and for allowing the inner peripheral slot to be located between the first blade member and the second blade member. The valve device includes a coupling member provided in the second side of the first blade member, and coupled between the first blade member and the second blade member, for coupling the first blade member and the second blade member together.

The piston includes an upper portion having an annular recess formed therein and arranged around the bore thereof, to form a peripheral wall between the annular recess and the bore thereof, and a sealing ring engaged into the annular recess of the piston.

A supporting base is further provided and includes a plate having an arm extended therefrom to support the cylinder housing, the cylinder housing includes a valve seat provided in the outlet tube and located between the outlet tube and the cylinder housing, and a spring-biased check valve is disposed in the outlet tube and engaged with the valve seat, to limit the pressurized air to flow from the chamber of the cylinder housing into the compartment of the outlet tube only, and to prevent the pressurized air from flowing backwardly from the compartment of the outlet tube into the chamber of the cylinder housing.

A motor is further provided and attached to the plate and includes a spindle extended through the plate, and an eccentric member coupled to the spindle of the motor and having a pin extended therefrom and coupled to the piston rod, to move the piston relative to the cylinder housing with the motor the eccentric member. The plate includes a gear rotatably attached thereto and having a space formed therein and defined by a peripheral casing, to receive and secure the eccentric member therein.

The cylinder housing includes a first duct, a second duct, and at least one third duct extended outwardly from the outlet tube and communicating with the compartment of the outlet tube, for receiving the pressurized air from the compartment of the outlet tube, a pressure gauge attached to the first duct, a nozzle coupled to the second duct, and a lid attached to the third duct.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a perspective of an air compressor in accordance with the present invention;

FIG. 2 is a partial cross sectional view of the air compressor, taken along lines 2-2 of FIG. 1;

FIG. 3 is a partial cross sectional view similar to FIG. 2, illustrating the operation of the air compressor;

FIG. 4 is a partial exploded view illustrating the piston and the valve member of the air compressor;

FIG. 5 is an enlarged perspective view illustrating the valve member for the air compressor;

FIG. 6 is a perspective view of the piston and the valve member of the air compressor;

FIG. 7 is a perspective view similar to FIG. 6, illustrating the operation of the air compressor; and

FIGS. 8, 9 are partial cross sectional views of the air compressor, taken along lines 8-8 and 9-9 of FIGS. 6 and 7 respectively.

### DETAILED DESCRIPTION OF THE PREFERRED EMBODIMENT

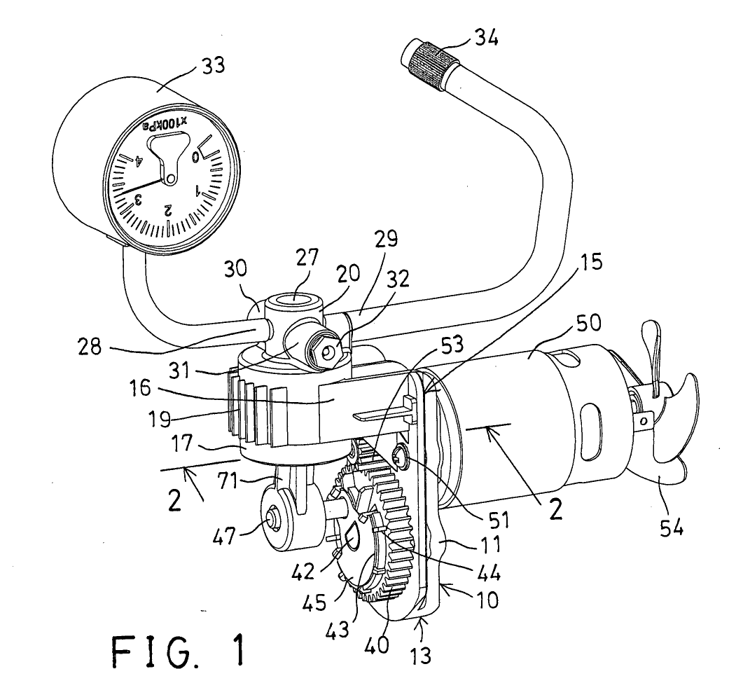

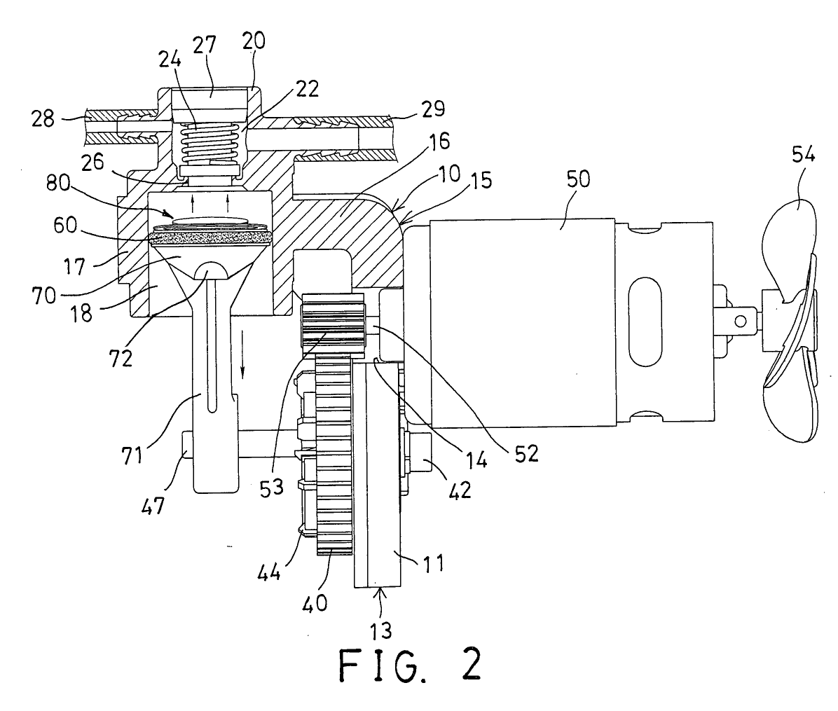

Referring to the drawings, and initially to FIGS. 1-3, an air compressor in accordance with the present invention comprises a supporting base 10 including a plate 11 having a lower portion 13, and having an orifice 14 formed in an upper portion 15 thereof (FIGS. 2, 3), and having an arm 16 laterally extended from the upper portion 15 thereof, and a cylinder housing 17 provided on or extended from the arm 16 and preferably formed integral with the arm 16 and the plate 11, best shown in FIGS. 2 and 3.

The cylinder housing 17 includes a chamber 18 formed therein (FIGS. 2-3), for slidably receiving a piston 70 therein, and includes one or more fins 19 extended laterally or radially outwardly therefrom (FIG. 1), for such as heat dissipating purposes. The piston 70 is slidably received in the chamber 18 of the cylinder housing 17, and includes an extension or piston rod 71 extended therefrom, for allowing the piston 70 to slide in reciprocating action in the chamber 18 of the cylinder housing 17, and to generate pressurized air.

The cylinder housing 17 includes an outlet tube 20 extended upwardly or outwardly from the top thereof, and having a compartment 22 formed therein and communicating with the chamber 18 of the cylinder housing 17, for receiving the pressurized air from the chamber 18 of the cylinder housing 17. A spring-biased check valve 24 (FIGS. 2, 3) may be disposed in the outlet tube 20, and engaged with a valve seat 26 that is formed or provided between the outlet tube 20 and the cylinder housing 17. A relief valve or safety valve (not shown) may further be provided and attached to the outlet tube 20, for relieving the pressurized air when the air pressure within the cylinder housing 17 and the outlet tube 20.

The spring-biased check valve 24 may thus be used to limit the pressurized air to flow from the chamber 18 of the cylinder housing 17 into the compartment 22 of the outlet tube 20 only, and to prevent the pressurized air from flowing backwardly from the compartment 22 of the outlet tube 20 into the chamber 18 of the cylinder housing 17. A cap 27 may be attached to the outer or free end of the outlet tube 20 with such as threading engagements, for blocking or enclosing the compartment 22 of the outlet tube 20, and for stably retaining the spring-biased check valve 24 within the compartment 22 of the outlet tube 20.

The cylinder housing 17 further includes one or more ducts 28, 29, 30, 31 extended outwardly from the outlet tube 20, and communicating with the compartment 22 of the outlet tube 20, for receiving the pressurized air from the compartment 22 of the outlet tube 20. The ducts 28, 29, 30, 31 may be coupled to various kinds of facilities that require pressurized air supplied thereto. For example, a relief valve 32 may further be provided and attached to the other ducts 31, for relieving the pressurized air when the air pressure within the cylinder housing 17 and/or the outlet tube 20 is over a predetermined pressure, and thus for preventing the cylinder housing 17 and/or the outlet tube 20 from being over-pressurized.

A pressure gauge 33 may be provided and attached to one of the ducts 28, for detecting and showing the air pressure within the cylinder housing 17 and/or the outlet tube 20. A nozzle 34 may be provided and attached to another duct 29, for allowing the pressurized air to be supplied from the chamber 18 of the cylinder housing 17 and the compartment 22 of the outlet tube 20 to various facilities that require pressurized air supplied thereto, with the nozzle 34.

It is to be noted that the provision or the extension of the ducts 28, 29, 30, 31 from the outlet tube 20 allows the pressure gauge 33 and the nozzle 34 and the relief valve 32 to be easily and readily attached to or coupled to the outlet tube 20 with the ducts 28, 29, 30, 31, without additional or specialized coupling members or tools. One or more lids (not shown) may further be provided and attached or secured to either of the ducts 28, 29, 30, 31 with such as threading engagements, for selectively enclosing or blocking the ducts 28, 29, 30, 31, when the ducts 28, 29, 30, 31 are not required to be used.

A gear 40 is rotatably attached to the lower portion 13 of the plate 11 with one or more bearings (not shown) and a shaft 42, and includes a space 43 formed therein and defined by a peripheral casing 44 for receiving and securing an eccentric member 45 therein (FIG. 1). The eccentric member 45 may be secured to the gear 40 with such as fasteners (not shown) and may thus be rotated in concert with the gear 40, and includes a crank or an eccentric pin 47 extended therefrom and coupled to the piston rod 71 of the piston 70, in order to actuate or to move the piston 70 relative to the cylinder housing 17 in reciprocating actions.

A motor 50 may be attached or secured to the upper portion 15 of the plate 11 with such as fasteners 51 (FIG. 1), and includes a spindle 52 extended through the upper orifice 14 of the plate 11 (FIGS. 2, 3), and includes a pinion 53 secured to the spindle 52 thereof, and engaged with the gear 40, for allowing the gear 40 to be rotated or driven by the motor 50 via the pinion 53, and thus for allowing the piston 70 to be actuated or moved relative to the cylinder housing 17 in reciprocating actions by the eccentric member 45 and the eccentric pin 47. A fan device 54 may further be provided and coupled to the motor 50 for being rotated or driven by the motor 50 to generate circulating or ventilating air.

In operation, as shown in FIGS. 2 and 3, the piston 70 may be actuated or moved relative to the cylinder housing 17 in reciprocating actions by the motor 50 via pinion 53, the gear 40, the eccentric member 45 and the eccentric pin 47, in order to generate a pressurized air, and to allow the pressurized air to flow into the outlet tube 20, and then to flow out through either or all of the ducts 28, 29, 30, 31, and thus to allow the air pressure within the cylinder housing 17 and/or the outlet tube 20 to be detected and shown by the pressure gauge 33, and to allow the pressurized air to be supplied into the facilities that require pressurized air supplied thereto, with the nozzle 34, and/or to the pressurized air to be relieved via the relief valve 32 when the cylinder housing 17 and/or the outlet tube 20 is over-pressurized.

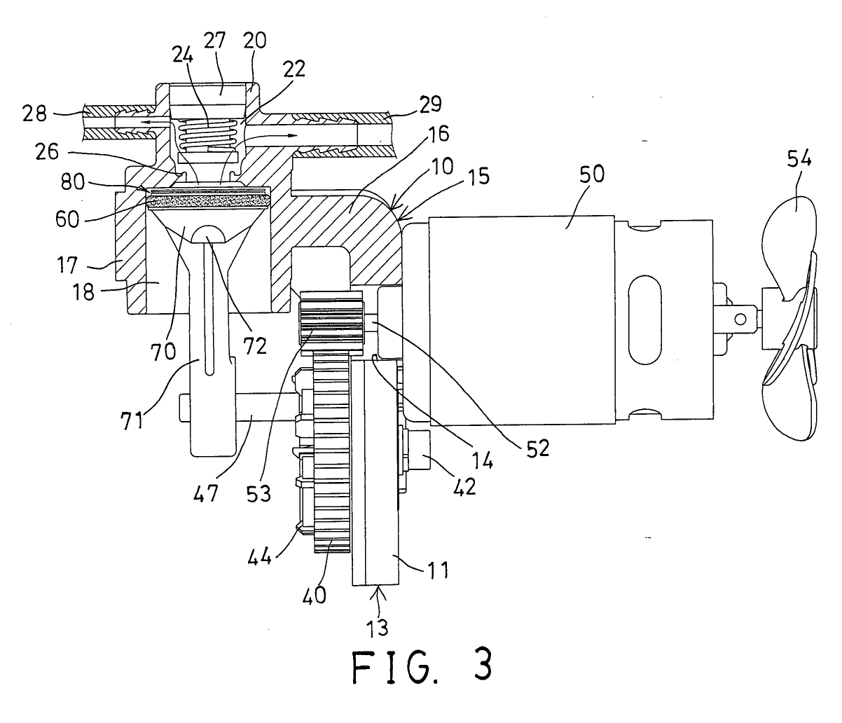

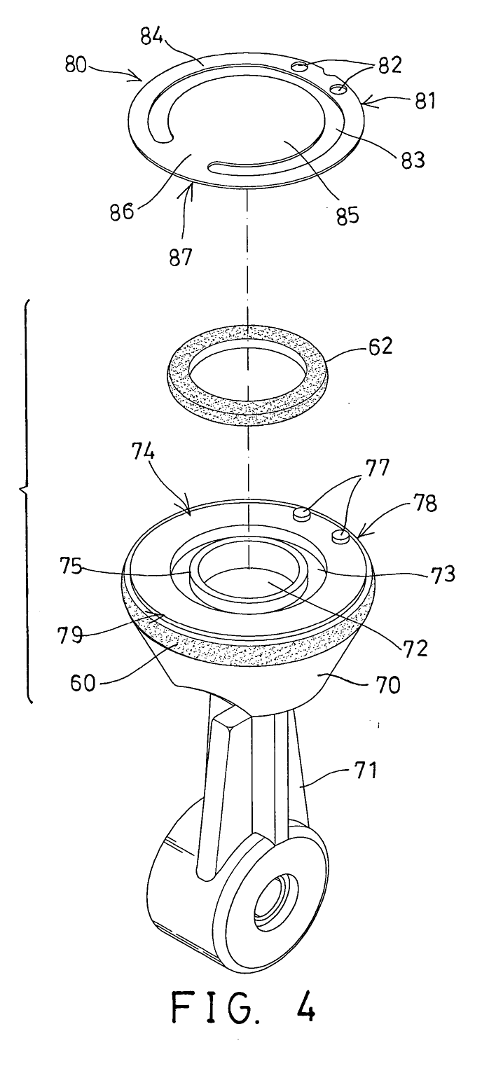

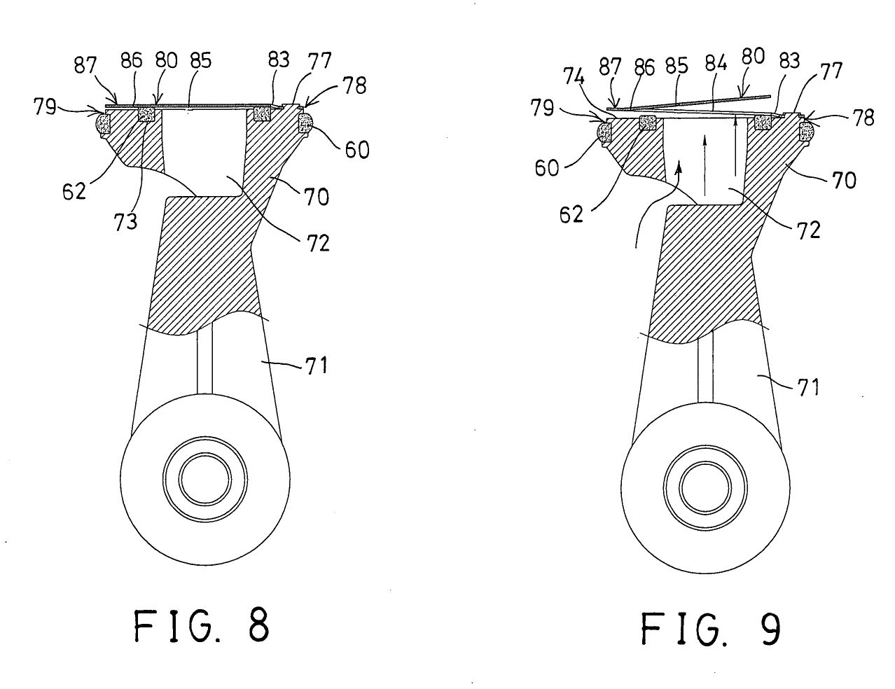

As shown in FIGS. 4-9, the piston 70 includes a sealing ring 60 attached or secured onto the outer peripheral portion thereof and slidably engaged with the cylinder housing 17, for making an air tight seal between the piston 70 and the cylinder housing 17; and includes a central bore 72 formed therein, and an annular recess 73 formed in the upper surface or portion 74 thereof and arranged around the central bore 72 thereof, to form or define an annular or peripheral wall 75 between the annular recess 73 and the central bore 72 thereof.

Another sealing ring 62 is engaged into the annular recess 73 of the piston 70, and preferably stably retained within the annular recess 73 of the piston 70 with the peripheral wall 75. The piston 70 further includes one or more pegs or anchoring members or rivets or catches 77 extended upwardly from one or first side 78 of the upper portion 74 or of the piston 70, and located opposite to the other or the second side 79 of the upper portion 74 or of the piston 70.

A valve device 80 includes one or first side 81 having one or more apertures 82 formed therein, for receiving the pegs or anchoring members or rivets or catches 77 of the piston 70, and thus for attaching or securing the one or first side 81 of the valve device 80 to the one or first side 78 of the piston 70. The valve device 80 further includes an inner peripheral slot 83 formed therein, for forming an outer peripheral blade member or first blade member 84 and an inner spring blade or second blade member 85 therein, and for allowing the inner peripheral slot 83 thereof to be located between the first blade member 84 and the second blade member 85.

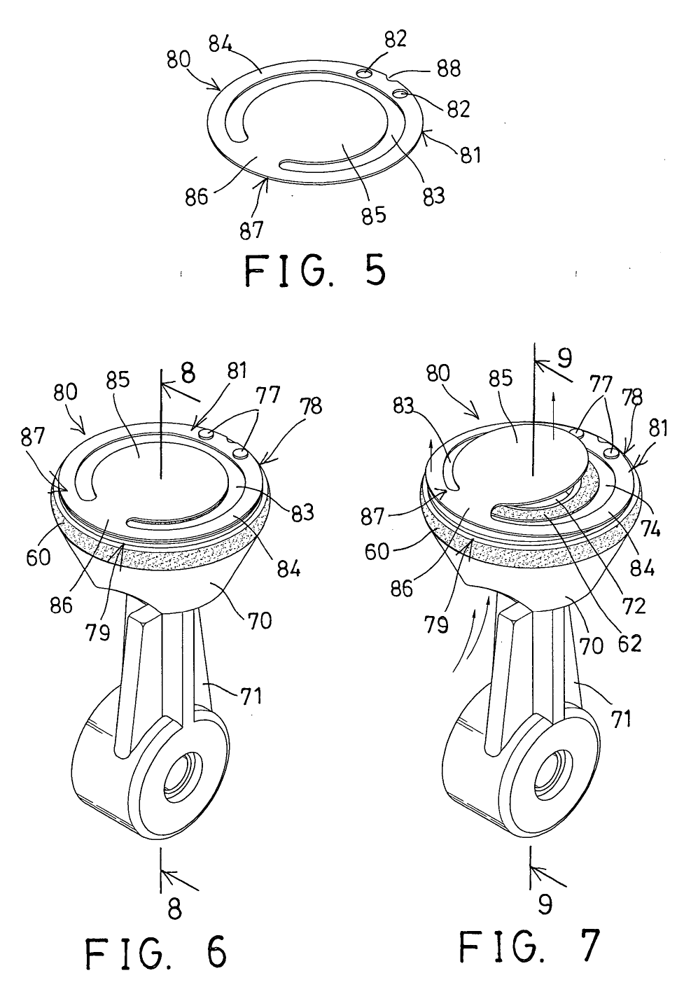

The valve device 80 further includes a coupling member 86 formed or provided in the other or the second side 87 thereof, and coupled between the first blade member 84 and the second blade member 85, for coupling the first blade member 84 and the second blade member 85 together. It is preferable that the valve device 80 further includes a notch 88 formed in the one or the first side 81 thereof (FIG. 5), for further increasing the bending or the resilience of the first blade member 84 and the second blade member 85 of the valve device 80.

In operation, as shown in FIGS. 6-9, the other or the second side 87 of the first blade member 84 or of the valve device 80 may be moved or sprung away from the upper portion 74 or of the piston 70, and the second blade member 85 of the valve device 80 may further be moved or sprung away from the other or the second side 87 of the first blade member 84 or of the valve device 80, for allowing the second blade member 85 of the valve device 80 to have a better movability or resilience to be moved or sprung away from the upper portion 74 or of the piston 70, in order to suitably and selectively engage with the sealing ring 62 and/or the peripheral wall 75 of the piston 70, and so as to openably and selectively block the central bore 72 of the piston 70, and so as to effectively control the air to flow through the central bore 72 of the piston 70.

Accordingly, the air compressor in accordance with the present invention includes an improved valve device for effectively controlling the air to flow through the valve device or to flow into and out of the cylinder housing.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. An air compressor comprising:
a cylinder housing including a chamber formed therein, and including an outlet tube having a compartment formed therein and communicating with said chamber of said cylinder housing, for receiving pressurized air from said chamber of said cylinder housing,
a piston slidably received in said chamber of said cylinder housing, and having a piston rod extended therefrom, and having a bore formed therein, and having a first side and a second side,
a motor coupled to said piston rod, for moving said piston relative to said cylinder housing in a reciprocating action, in order to generate pressurized air, and
a valve device including a first blade member having a first side secured to said first side of said piston, and having a second side opposite to said first side thereof, and a second blade member coupled to said second side of said first blade member, for blocking said bore of said piston, and for increasing a resilience of said second blade member of said valve device relative to said piston.

2. The air compressor as claimed in claim 1, wherein said valve device includes at least one aperture formed in said first side of said first blade member, and said piston includes at least one catch extended upwardly from said first side thereof and engaged into said at least one aperture of said first blade member, for attaching said first blade member to said first side of said piston.

3. The air compressor as claimed in claim 1, wherein said valve device includes a notch formed in said first side thereof, for increasing a resilience of said first blade member.

4. The air compressor as claimed in claim 1, wherein said valve device includes an inner peripheral slot formed therein, for forming said first blade member and said second blade member therein, and for allowing said inner peripheral slot to be located between said first blade member and said second blade member.

5. The air compressor as claimed in claim 4, wherein said valve device includes a coupling member provided in said second side of said first blade member, and coupled between said first blade member and said second blade member, for coupling said first blade member and said second blade member together.

6. The air compressor as claimed in claim 1, wherein said piston includes an upper portion having an annular recess formed therein and arranged around said bore thereof, to form a peripheral wall between said annular recess and said bore thereof, and a sealing ring engaged into said annular recess of said piston.

7. The air compressor as claimed in claim 1 further comprising a supporting base including a plate having an arm extended therefrom to support said cylinder housing, said cylinder housing including a valve seat provided in said outlet tube and located between said outlet tube and said cylinder housing, and a spring-biased check valve disposed in said outlet tube and engaged with said valve seat, to limit the pressurized air to flow from said chamber of said cylinder housing into said compartment of said outlet tube only, and to prevent the pressurized air from flowing backwardly from said compartment of said outlet tube into said chamber of said cylinder housing.

8. The air compressor as claimed in claim 7 further comprising a motor attached to said plate and including a spindle extended through said plate, and an eccentric member coupled to said spindle of said motor and having a pin extended therefrom and coupled to said piston rod, to move said piston relative to said cylinder housing with said motor said eccentric member.

9. The air compressor as claimed in claim 8, wherein said plate includes a gear rotatably attached thereto and having a space formed therein and defined by a peripheral casing, to receive and secure said eccentric member therein.

10. The air compressor as claimed in claim 1, wherein said cylinder housing includes a first duct, a second duct, and at least one third duct extended outwardly from said outlet tube and communicating with said compartment of said outlet tube, for receiving the pressurized air from said compartment of said outlet tube, a pressure gauge attached to said first duct, a nozzle coupled to said second duct, and a lid attached to said at least one third duct.
